(19) United States
(12) Patent Application Publication
(10) Pub. No.: US 2018/0232054 A1
(43) Pub. Date: Aug. 16, 2018

(54) METHOD AND SYSTEM FOR DETERMINING DRUG INTAKE BY A SUBJECT BY MONITORING GESTURES OF SUBJECT

(71) Applicant: WIPRO LIMITED, Bangalore (IN)

(72) Inventor: Rajeev P. NAIR, Bangalore (IN)

(73) Assignee: Wipro Limited.

(21) Appl. No.: 15/469,980

(22) Filed: Mar. 27, 2017

(30) Foreign Application Priority Data
Feb. 10, 2017 (IN) 201741004905

(51) Int. Cl. G06F 3/01; A61B 5/00; A61B 5/11; G06F 19/00; G06K 9/00

(52) U.S. Cl. CPC G06F 3/017; A61B 5/0002; A61B 5/11; G06F 19/34; G06K 9/00335; G06K 9/00375; G06F 19/30; G06F 19/3418

(57) ABSTRACT

The present disclosure relates to a method and system for determining drug intake by subject by monitoring gestures of subject by gesture monitoring system. The gesture monitoring system receives plurality of sensor data from registered device worn on finger of both hands of a subject, determine parameters from plurality of sensor data, comprising magnitude change, turn angle change, direction change and time difference between consecutive samples of plurality of sensor data, determines one or more events associated with finger of both hands of subject by comparing parameters with pre-defined range of turn angle change, direction change and time difference, correlate one or more events of finger of both hands occurring at same time duration using predefined rules to identify sequences of intermediate gestures and identify one or more final gestures by correlating one or more sequences of intermediate gestures using the pre-defined rules for determining drug intake by subject.

### TECHNICAL FIELD

The present subject matter is related in general to the gesture monitoring system, more particularly, but not exclusively, to a method and system for determining drug intake by a subject by monitoring gestures of the subject.

### BACKGROUND

In recent years, advancement in computing technologies has greatly influenced and modernized healthcare system. One of the key essentials of the healthcare system is adherence to medical drug, for medical therapy to be successful or for a clinical trial to be effective. The aim of any prescribed medical therapy is to achieve certain desired results in patients concerned. However, despite of all best efforts on the part of the healthcare professionals, the results might not be achievable if the patients are non-compliant. The non-compliance may have serious effects from the perspective of disease management. Hence, compliance to medical drug has been a topic of clinical concern due to the widespread nature of non-compliance.

Today, there are many devices for monitoring drug intake compliance of the patients. However, there are no fool proof and reliable methods to check the adherence level of the drug intake. The non-adherence to drug/medication leads to several serious issues such as recurrence of illness as the past therapy was followed partially, capture of erroneous clinical trial data which may lead to drug recall, loss of revenues for pharmaceutical companies, increase in government spending on health insurance and the like.

The information disclosed in this background of the disclosure section is only for enhancement of understanding of the general background of the invention and should not be taken as an acknowledgement or any form of suggestion that this information forms the prior art already known to a person skilled in the art.

### SUMMARY

In an embodiment, the present disclosure relates to a method of determining drug intake by a subject by monitoring gestures of the subject. The method comprising receiving a plurality of sensor data from a registered device worn on a finger of both hands of the subject. The plurality of sensor data is sensed for a pre-defined time duration after providing an alert to the subject. The method comprising determining parameters from the plurality of sensor data. The parameters comprise magnitude change, turn angle change, direction change and time difference between consecutive samples of the plurality of sensor data. The method comprising determining one or more events associated with the finger of both hands of the subject by comparing the parameters with a pre-defined range of turn angle change, direction change and time difference, correlating the one or more events of the finger of both the hands occurring at same time duration using predefined rules to identify one or more sequences of intermediate gestures and identifying one or more final gestures by correlating the one or more sequences of intermediate gestures using the pre-defined rules for determining drug intake by the subject.

In an embodiment, the present disclosure relates to a gesture monitoring system for determining drug intake by a subject by monitoring gestures of the subject. The gesture monitoring system comprises a processor and a memory communicatively coupled to the processor, wherein the memory stores processor executable instructions, which, on execution, may cause the gesture monitoring system to receive a plurality of sensor data from a registered device worn on a finger of both hands of the subject. The plurality of sensor data is sensed for a pre-defined time duration after providing an alert to the subject. The gesture monitoring system determines parameters from the plurality of sensor data. The parameters comprise magnitude change, turn angle change, direction change and time difference between consecutive samples of the plurality of sensor data. The gesture monitoring system determines one or more events associated with the finger of both hands of the subject by comparing the parameters with a pre-defined range of turn angle change, direction change and time difference, correlates the one or more events of the finger of both the hands occurring at same time duration using predefined rules to identify one or more sequences of intermediate gestures and identifies one or more final gestures by correlating the one or more sequences of intermediate gestures using the pre-defined rules for determining drug intake by the subject.

In an embodiment, the present disclosure relates to a non-transitory computer readable medium including instructions stored thereon that when processed by at least one processor may cause a gesture monitoring system to receive a plurality of sensor data from a registered device worn on a finger of both hands of the subject, where the plurality of sensor data is sensed for a pre-defined time duration after providing an alert to the subject, determine parameters from the plurality of sensor data, where the parameters comprise magnitude change, turn angle change, direction change and time difference between consecutive samples of the plurality of sensor data, determine one or more events associated with the finger of both hands of the subject by comparing the parameters with a pre-defined range of turn angle change, direction change and time difference, correlate the one or more events of the finger of both the hands occurring at same time duration using predefined rules to identify one or more sequences of intermediate gestures and identify one or more final gestures by correlating the one or more sequences of intermediate gestures using the pre-defined rules for determining drug intake by the subject.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1a illustrates an exemplary environment for determining drug intake by a subject by monitoring gestures of the subject in accordance with some embodiments of the present disclosure;

FIG. 1b illustrates an exemplary embodiment of a user wearable device in accordance with some embodiments of the present disclosure;

FIG. 2 shows a detailed block diagram of a gesture monitoring system in accordance with some embodiments of the present disclosure;

FIG. 3 shows an exemplary representation of determining drug intake by a subject by monitoring gestures of the subject in accordance with some embodiments of the present disclosure;

FIG. 4 illustrates a flowchart showing a method of determining drug intake by a subject by monitoring gestures of the subject in accordance with some embodiments of present disclosure; and

FIG. 5 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

The present disclosure may relate to a method and a gesture monitoring system for determining drug intake by a subject by monitoring gestures of the subject. In an embodiment, the present disclosure may determine drug intake by the subject by determining one or more gestures of the subject based on pre-defined rules. To determine drug intake by the subject, the gesture monitoring system may receive a plurality of sensor data from a registered device worn by the subject on a finger of both hands. In an embodiment, prescription data associated with the subject may be received either from a clinical system or a user device. At an appropriate time based on the prescription data, a vibrator configured within the registered wearable device alerts the subject to the take the drug dosage and identifies whether the subject indeed takes the drug based on gestures of finger of both the hands. The gestures may be identified by correlating one or more events associated with finger of both the hands of the subject based on the pre-defined rules. In the present disclosure, the use of natural gestures of the subject, which may not be easily faked are analysed to confirm on the actual intake of the drug.

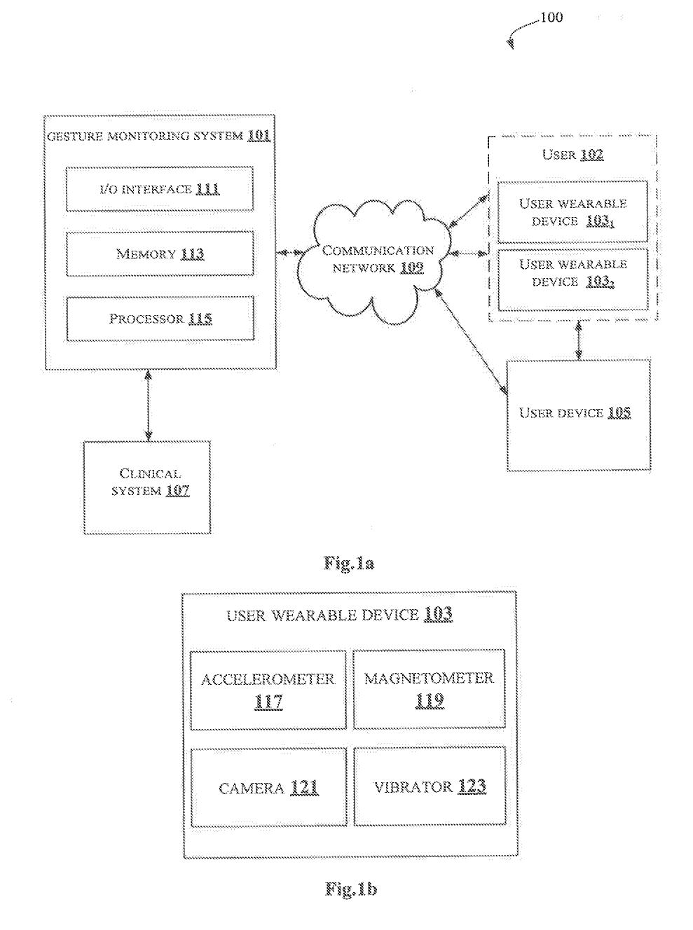

FIG. 1a illustrates an exemplary environment for determining drug intake by a subject by monitoring gestures of the subject in accordance with some embodiments of the present disclosure.

As shown in FIG. 1a, the environment 100 comprises a gesture monitoring system 101 connected through a communication network 109 to a user wearable device 103₁ and a user wearable device 103₂ (collectively called as user wearable device 103) of the subject (or the user) 102 and a user device 105. The gesture monitoring system 101 may also be connected to a clinical system 107. In an embodiment, the clinical system 107 may be connected to the gesture monitoring system 101 through the communication network 109. In an embodiment, the user wearable device 103₁ and the user wearable device 103₂ may be worn on a finger of both hands of the subject 102. For instance, the user wearable device 103₁ may be worn on left index finger and the user wearable device 103₂ may be worn on the right index finger of the hands of the subject 102.

FIG. 1b illustrates an exemplary embodiment of a user wearable device in accordance with some embodiments of the present disclosure. As shown in FIG. 1b, the user wearable device 103 comprises a three-dimensional accelerometer 117, a magnetometer 119, a camera 121 and a vibrator 123. The user wearable device 103 may have a communication module (not shown explicitly in FIG. 1b) to communicate with another device through wireless medium. In an embodiment, the user wearable device 103 may be a ring worn by the subject 102 on a finger of both hands. In an embodiment, the three-dimensional accelerometer 117 collects coordinate data associated with the finger of each hands of the subject 102 and the magnetometer 119 may be used to determine orientation details associated with the finger of both the hands the subject 102. In an embodiment, the three-dimensional accelerometer 117 and the magnetometer 119 may be activated on receiving an activation signal from the gesture monitoring system 101. The three-dimensional accelerometer 117 and the magnetometer 119 may remain active in low power mode for a pre-determined time duration until a motion of the subject 102 is detected. In an instance, on detecting the motion of the subject 102, the three-dimensional accelerometer 117 and the magnetometer 119 become active in high power mode and acquire plurality of sensor data from both the hands of the subject 102. Further, the vibrator 123 may be initiated on receiving the activation signal. The vibrator 123 may be used for providing vibration signals to the finger of both the hands of the subject 102 for notifying the subject 102 to consume the drug. In an embodiment, the three-dimensional accelerometer 117 and the magnetometer 119 may be activated when the vibrator is activated by an activation signal. The vibrator 123 may vibrate and alert the subject 102 for a pre-defined time duration as configured by the subject 102. The vibrator 123 alerts the subject 102 based on the prescription data received by the clinical system 107 and the user device 105. In an embodiment, vibration signals to the subject 102 may be re-initiated at pre-configured time intervals. The vibration signal to the subject 102 continues until the subject 102 takes the drug or number of alerts provided to the subject 102 exceeds a maximum configured limit. Further, the camera 121 present in the user wearable device 103 may capture images of the drugs being taken by the subject 102. In an embodiment, the images may help to ascertain whether correct drug has been taken by the subject 102.

Returning to FIG. 1a, the user device 105 may include, but is not limited to, a laptop, a desktop computer, a Personal Digital Assistant (PDA), a notebook, a smartphone, a tablet and any other computing devices. A person skilled in the art would understand that any other user devices which are not mentioned explicitly, but are capable to communicate data, may also be included in the present disclosure. In an embodiment, the user device 105 may register the user wearable device 103 with the gesture monitoring system 101 using an application. In an embodiment, the subject 102 may pre-configure a time for drug intake and a time for receiving alert through the user device 105. In addition, the subject 102 may also pre-configure a duration for repeating the alert generation through the user device 105 when the subject 102 ignores the initial alert. In an embodiment, the user device 105 may also facilitate uploading of scanned prescription data to the gesture monitoring system 101. In an embodiment, the user device 105 may also generate alerts for drug intake to the subject 102, where the drug related details may be displayed on a display screen of the user device 105. The gesture monitoring system 101 determines drug intake by the subject 102. In an embodiment, the gesture monitoring system 101 may include, but are not limited to, a cloud server, a laptop, a desktop computer, a Personal Digital Assistant (PDA), a notebook, a smartphone, a tablet and any other computing devices.

To determine drug intake by the subject, the gesture monitoring system 101 may receive the plurality of sensor data from the user wearable device 103. In an embodiment, the plurality of sensor data may be sensed for a pre-defined time duration after providing the activation signal to the subject. The activation signal may be transmitted depending on the prescription data which may be received by the user device 105 and the clinical system 107. The clinical system 107 may provide prescription details associated with the subject 102 such as, drug details, exact dosage, time of drug intake and the like to the gesture monitoring system 101. A person skilled in the art would understand that any other type of prescription data not mentioned explicitly may also be included in the present disclosure. On receiving the plurality of sensor data, the gesture monitoring system 101 determines parameters of the plurality of sensor data such as, magnitude change, turn angle change, direction change and time difference between consecutive samples of the plurality of sensor data. Further, the parameters associated with the plurality of sensor data may be compared with a pre-defined range of turn angle change, direction change and time difference. Based on the comparison, the gesture monitoring system 101 determines one or more events associated with the finger of both hands of the subject 102. In an embodiment, the one or more events may comprise clockwise motion, anti-clockwise motion, angular motion, rotary motion, tilt movement and linear motion of at least one hand of the subject 102. A person skilled in the art would understand that any other motion associated with hands of the subject which is not mentioned explicitly may also be included in the present disclosure. Further, the gesture monitoring system 101 may identify one or more sequences of intermediate gestures of the subject by correlating the one or more events which may be occurring at the same time. The one or more events may be correlated using pre-defined rules. Subsequently, the gesture monitoring system 101 determines drug intake by the subject by identifying one or more final gestures by correlating the one or more sequences of intermediate gestures using the pre-defined rules. In an embodiment, if the drug is not taken by the subject 102 after multiple alerts, then such an act may be regarded as non-adherence to drug intake.

The gesture monitoring system 101 comprises an I/O Interface 111, a memory 113 and a processing unit 115. The I/O interface 111 may be configured to receive the plurality of sensor data from the user wearable device 103 worn on the finger of both hands of the subject 102. The I/O interface 111 may also receive dosage data from the user device 105 and the clinical system 107.

The received information from the I/O interface 111 is stored in the memory 113. The memory 113 is communicatively coupled to the processing unit 115 of the gesture monitoring system 101. The memory 113 also stores processor instructions which cause the processing unit 115 to execute the instructions for determining drug intake by the subject, 102.

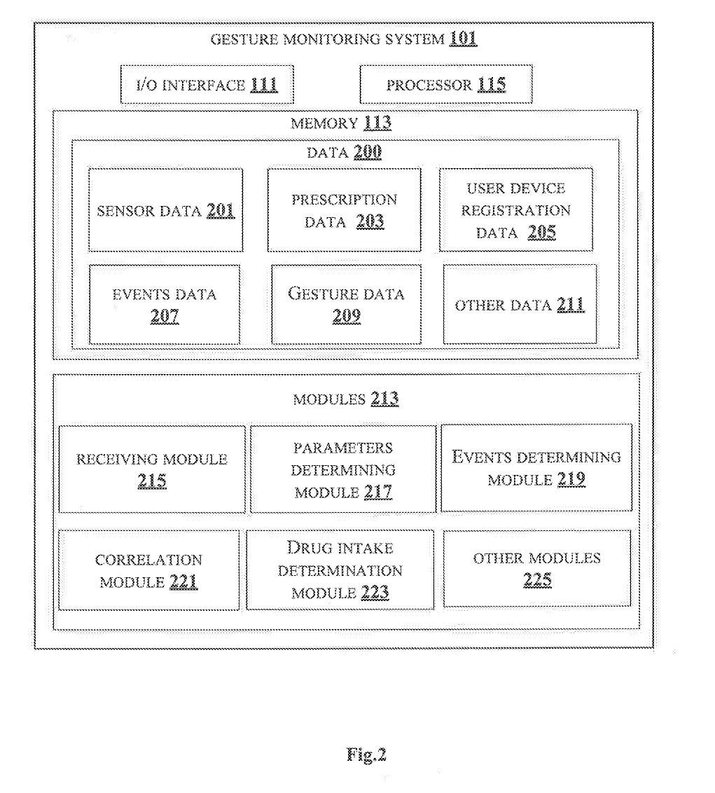

FIG. 2 shows a detailed block diagram of a gesture monitoring system in accordance with some embodiments of the present disclosure.

Data 200 and one or more modules 213 of the gesture monitoring system 101 are described herein in detail. In an embodiment, the data 200 comprises sensor data 201, prescription data 203, user device registration data 205, events data 207, gestures data 209 and other data 211.

The sensor data 201 may include a plurality of samples of coordinate details associated with finger of both hands of the subject 102 and the orientation details associated with the finger of both hands of the subject 102 along with a time duration for each of the plurality of samples. The coordinate details may be received from the three-dimensional accelerometer 117 and the orientation data from the magnetometer 119. The sensor data 201 may be received upon providing the alert to the user wearable device 103. The sensor data 201 may be collected for the pre-defined time duration. Further, the sensor data 201 may be analyzed to determine parameters associated with each of the sensor data. The parameters may include magnitude change, turn angle change, direction change and time difference between consecutive samples of the plurality of sensor data.

The prescription data 203 may include details regarding the prescription provided to the subject 102 by doctor or any medical practitioner. The details may include, but are not limited to, drug details, exact dosage, time of the intake and the like. The prescription data 203 may be received from the clinical system 107 or from the user device 105.

The user device registration data 205 may include registration details associated with the user wearable device 103. In an embodiment, the registration details may include details of the subject 102 such as, subject name, age, health details and the like. A person skilled in the art would understand that any other details associated with the subject not mentioned explicitly may also be included in the present disclosure. The user device registration data 205 may also comprise details about the user wearable device 103 worn by the subject 102. In an embodiment, the user wearable device 103 details may include model number, unique identification number and the like. A person skilled in the art would understand that any other details associated with the user wearable device, not mentioned explicitly may also be considered in the present disclosure.

The events data 207 may include details regarding the one or more events determined from the finger of both hands of the subject 102. In an embodiment, the one or more events comprises clockwise motion, anti-clockwise motion, angular motion, rotary motion, tilt movement and linear motion of both hands of the subject 102. For example, clockwise motion in zy plane from clock degree between 10 to 2 degrees, speed of movements of each hand of the subject 102, anticlockwise movement in zy plane from clock degree between 10 to 2 degrees, linear movement in xy plane with orientation of 45 degrees from magnetic north, upward/downward angular movement in zy plane with some angular velocity. The events data 207 may also include the occurrence period for each of the events. The occurrence period may be the time when each of the events occur.

The gesture data 209 may include details regarding the one or more sequences of intermediate gestures identified from the one or more events of the fingers of both hands of the subject 102. The one or more sequences of intermediate gestures may be identified using the pre-defined rules which may be a formal grammar. In an embodiment, the one or more sequences of intermediate gestures may be identified based on an order of time duration. The gesture data 209 may also include one or more final gestures such as, tearing a drug strip, sliding of drug from strip, taking the drug to mouth, placing the drug in the mouth, holding a container, tilting the container, opening and closing of a tap, holding a container of water and taking the container of water to mouth and the like. The one or more final gestures may be identified by correlating the one or more sequences of intermediate gestures using the pre-defined rules.

The other data 211 may store data, including temporary data and temporary files, generated by modules 213 for performing the various functions of the gesture monitoring system 101. In an embodiment, the other data 211 may include the pre-defined range of the turn angle change, direction change and time difference.

In an embodiment, the data 200 in the memory 113 are processed by the one or more modules 213 of the gesture monitoring system 101. As used herein, the term module refers to an application specific integrated circuit (ASIC), an electronic circuit, a field-programmable gate arrays (FPGA), Programmable System-on-Chip (PSoC), a combinational logic circuit, and/or other suitable components that provide the described functionality. The said modules when configured with the functionality defined in the present disclosure will result in a novel hardware.

In one implementation, the one or more modules 213 may include, but are not limited to, a receiving module 215, parameters determining module 217, an event determining module 219, a correlation module 221 and a drug intake determination module 223. The one or more modules 213 may also comprise other modules 225 to perform various miscellaneous functionalities of the gesture monitoring system 101. It will be appreciated that such modules 213 may be represented as a single module or a combination of different modules.

The receiving module 215 may receive the plurality of sensor data from the user wearable device 103 on activation of the user wearable device 103. In an embodiment, the receiving module 215 may receive still images of the subject 102 from the camera 121. In an embodiment, the plurality of sensor data received may be filtered by the user wearable device 103 to provide valid sensor data. For example, the sensor data may be valid if delta values of the coordinate data of the finger of both hands of the subject 102 is within a pre-defined range and pre-defined time duration. The receiving module 215 may also receive the registration details associated with the user wearable device 103 from the user device 105. The receiving module 215 may also receive the prescription data either from the user device 105 or from the clinical system 107.

The parameters determining module 217 may determine the parameters from the plurality of sensor data. The parameters may include magnitude change, turn angle change, direction change and time difference between consecutive samples of the plurality of sensor data. The parameter determining module 217 may determine magnitude change in the values of coordinates data such as (x, y, z) coordinates of the fingers of both the hands and the time difference between the consecutive samples of the plurality of sensor data. In an embodiment, the parameters determining module 217 may determine the turn angle and direction using direction cosines when the time difference and the magnitude change is less than a pre-defined range. For example, if time difference is less than 150 ms and magnitude change is less than and equal to abs (5), the parameter determining module 217 may determine the turn angle and the direction associated with each of the plurality of sensor data.

The events determining module 219 may determine one or more events of the finger of both hands of the subject 102. The events determining module 219 may determine one or more events by comparing the parameters determined with the pre-defined range of turn angle change, direction change and time difference of a trained sequence of gestures. The events determining module 219 may also determine the occurrence period associated with each event. Examples of one or more events include, clockwise motion of a finger of the subject 102 in zy plane from clock degree between 10 to 2 degrees, speed of movement for each event, anticlockwise movement of the finger of the subject 102 in zy plane from clock degree between 10 to 2 degrees, linear movement of the finger of the subject 102 in xy plane with orientation of 45 degrees from magnetic north, upward/downward angular movement of the finger of the subject 102 in zy plane with some angular velocity and the like.

The correlation module 221 may pair the one or more events of the finger of both hands of the subject 102 which are occurring at the same time based on the occurrence period. In an embodiment, the one or more events may be paired, if a difference between the occurrence time of the one or more events is less than one second. For example, the one or more events of left hand may be paired to the one or more events of right hand occurring at the same time. In an embodiment, the one or more paired events may be referred as relative events. Further, in case, any event, which may not be paired to any other event based on same time, then the correlation module 221 pairs the event with a null event. The correlation module 221 uses the pre-defined rules for pairing. The correlation module 221 may pair the one or more events to identify one or more sequences of intermediate gestures. Below are some examples of determining intermediate gestures:

An event from the user wearable device 103 as clockwise event from user wearable device 103₁ with occurrence period (t₀, 450 ms), an anti-clockwise event from user wearable device 103₂ with occurrence period (t₀, 400 ms) may get identified as medical strip tearing gesture.

An angular motion in zy plane from wearable device 103₁ with occurrence period (t₀+500 ms, 750 ms) and null events between t₀+500 ms and t₀+1250 ms at the wearable device 103₂ may get identified as transient medicine popping gesture.

An angular motion in zy plane at wearable device 103₁ with occurrence period (t₀+800 ms, 750 ms) and null events between t₀+800 ms and t₀+1550 ms at wearable device 103₂ may get identified as medicine popped gesture.

The drug intake determination module 223 may determine drug intake by the subject 102 by identifying one or more final gestures associated with the fingers of both hands of the subject 102 by correlating the one or more sequences of the intermediate gestures using the pre-defined rules. The one or more final gestures are identified by correlating the one or more sequences of intermediate gestures using the pre-defined rules. In an embodiment, the pre-defined rules may contain sequence of events and time duration at which the event may occur.

Below are some examples of correlation of the one or more sequences of gestures to identify one or more final gestures using the predefined rules are:

Gesture of Medicine in-Take:

Gesture of popping medicine into mouth+(ground linear movement)*@t<=1000 ms+gesture of drinking @1000<=t<=activity period−10000 ms 1

Gesture of Popping Medicine into Mouth:

Medical strip tearing+transient medicine popping @t<=3000 ms+medicine popped @t<=2000 ms|transient medicine popping+medicine popped @t<=2000 ms 2

Gesture of Drinking Water:

Tap movement+hand movement @t<=3000 ms|tilt movement+onair movement @t<=1000 ms+tilt movement @t<=5000 ms+onair movement @t<=5000 ms+hand movement @t<=2000 ms 3

Medical Strip Tearing:

Clockwise event in zy plane at user wearable device 103₁, clock degree (>=10° to <=2°), duration<=450 ms+anti-clockwise event in zy plane at user wearable device 103₂, clock degree (>=10° to <=2°), duration<=450 ms@t<=10 ms 4

Transient Medicine Popping:

Upward angular movement in zy plane in user wearable device 103₁, angular velocity (<=0.5 rads/s), duration<=750 ms|upward angular movement in zy plane at user wearable device 103₂, angular velocity (<=0.5 rads/s), duration<=750 m 5

Medicine Popped:

Downward angular movement in zy plane at user wearable device 103₁, angular velocity (<=0.5 rads/s), duration<=750 ms|downward angular movement in zy plane user at wearable device 103₂, angular velocity (<=0.5 rads/s), duration<=750 ms 6

Tap Movement:

Clockwise event in xy plane user at wearable device 103₂, clock degree (>=10° to <=2°), duration<=450 ms+anti-clockwise event in xy plane user wearable device 103₁, clock degree (>=10° to <=2°), duration<=450 ms@t<=5000 ms|clockwise event in xy plane at user wearable device 103₂, clock degree (>=10° to <=2°), duration<=450 ms+anti-clockwise event in xy plane at user wearable device 103₂, clock degree (>=10° to <2°), duration<=450 ms@t<=5000 ms 7

Hand Movement:

Upward angular movement event in zy plane at user wearable device 103₁, angular velocity (<=0.5 rads/s)+downward angular movement event in zy plane at user wearable device 103₁, angular velocity (<=0.5 rads/s) @t<=3000 ms|upward angular movement event in zy plane user wearable device 103₂, angular velocity (<=0.5 rads/s)+downward angular movement event in zy plane at user wearable device 103₂, angular velocity (<=0.5 rads/s) @t<=3000 ms 8

Tilt Movement:

Anti-clockwise event in zy plane at user wearable device 103₁, clock degree (>=10° to <=2°), duration<=1450 ms, angular velocity (<=0.01 rads/s)|anti-clockwise event in zy plane at user wearable device 103₂, clock degree (>=10° to <=2°), duration<=1450 ms, angular velocity (<=0.01 rads/s) 9

Ground Linear Movement:

Linear event in (xy plane at user wearable device 103₁, magnetic north orientation degree (o), duration<=activity period−10000 ms+linear event in xy plane at user wearable device 103₂, magnetic north orientation degree (o), duration<=activity period−10000 ms@t<=1 ms)+→one or more linear events 10

Linear Movement:

Linear event in zy/zx plane at user wearable device 103₁, magnetic north orientation degree (0°), duration<=5000 ms|linear event in zy/zx plane at user wearable device 103₂, magnetic north orientation degree (0°), duration<=5000 ms 11

In the above example, the symbol (*) may indicate zero or more occurrence, symbol (+) may indicate at least one occurrence, (@t) indicates a delta time that the associated event may occur after the occurrence of the preceding event.

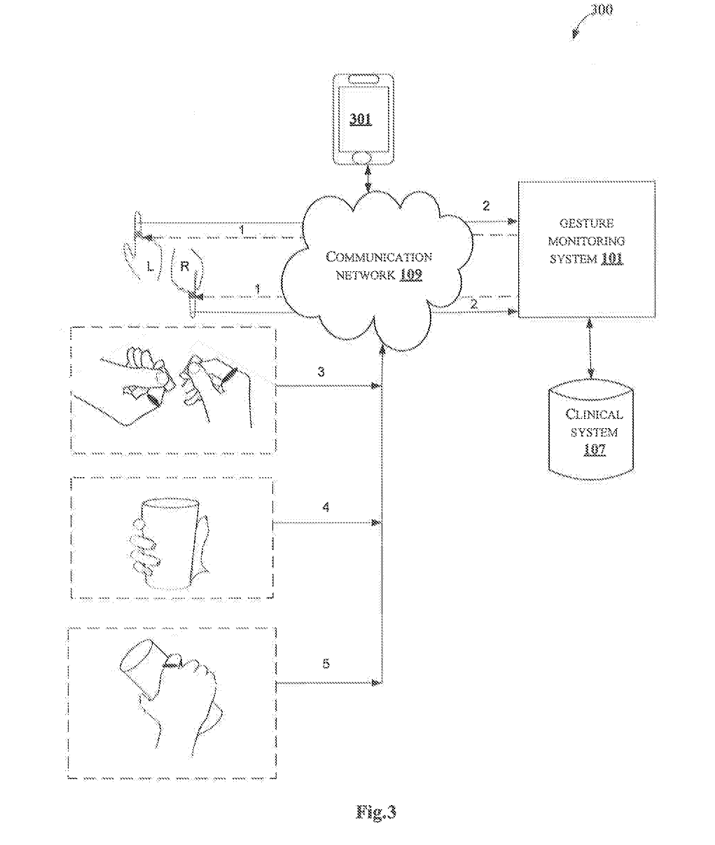

FIG. 3 shows an exemplary representation of determining drug intake by a subject by monitoring gestures of the subject data in accordance with some embodiments of the present disclosure.

As shown in FIG. 3, the environment 300 illustrates an exemplary representation of determining drug intake by the subject 102. The environment 300 comprises the gesture monitoring system 101 connected to a mobile phone 301 and the clinical system 107 through the communication network 109. Further, as shown in FIG. 3, the gesture monitoring system 101 is connected to user wearable device worn on index fingers, represented as left (L) and right (R) of both hands of the subject 102. A person skilled in the art would understand that the user wearable device may be worn on any fingers of the hands in the present disclosure. Initially, at step (1), the gesture monitoring system 101 may transmit an activation signal to the user wearable device worn on the left and right fingers to alert the subject 102 for drug intake. The activation signal may be transmitted based on the prescription data received previously by the gesture monitoring system 101. On receiving the activation signal, the user wearable device of both the fingers may initiate the vibrator 123 to alert the subject 102. In an embodiment, the user wearable device of both fingers may vibrate for a pre-defined time duration as configured by the subject 102. In an embodiment, the user wearable device 103 of both the fingers may remain in a low power mode for a pre-determined time duration until a movement from at least one hand is detected. Once the movement is detected, the user wearable device switches to high power mode and collects hand gesture data. In an embodiment, the details regarding the drug may be provided through the mobile phone 301. The subject 102 may take the drug based on details provided by the mobile phone 301. In case, the subject 102 ignores the alert signal provided by the user wearable device 103 of both the fingers, the vibration may resume after a pre-defined time interval. In an embodiment, if the movement from at least one finger of the subject 102 is not detected within the pre-determined time, the gesture monitoring system 101 may determine that the subject 102 ignored the alert signal. In an embodiment, the subject 102 may also configure snooze duration for the alert. Once, the movement of at least one finger is sensed, the plurality of sensor data is collected for the pre-determined time duration. Then, the plurality of sensor data associated with the movement is provided by at least one of the user wearable device 103 to the gesture monitoring system 101 as shown in step (2). The plurality of sensor data may include data as shown in step 3, 4 and 5. Step 3, 4 and 5 illustrate exemplary embodiment gestures of drug intake by the subject 102 on receiving the alert. Step (3) shows hands of the subject 102 tearing the drug strip, which involves clockwise and anticlockwise rotation of the hands. Step (4) shows a hand of the subject 102 holding a glass of water. Step (5) shows the hand of the subject 102 drinking the water. The hand is in a position of upward direction at some angle. The data from each of the steps 3, 4 and 5 are received by the gesture monitoring system 101 for determining the drug intake by the subject 102. The gesture monitoring system 101 may analyse the data and deduce gestures which may adhere to drug intake by the subject 102.

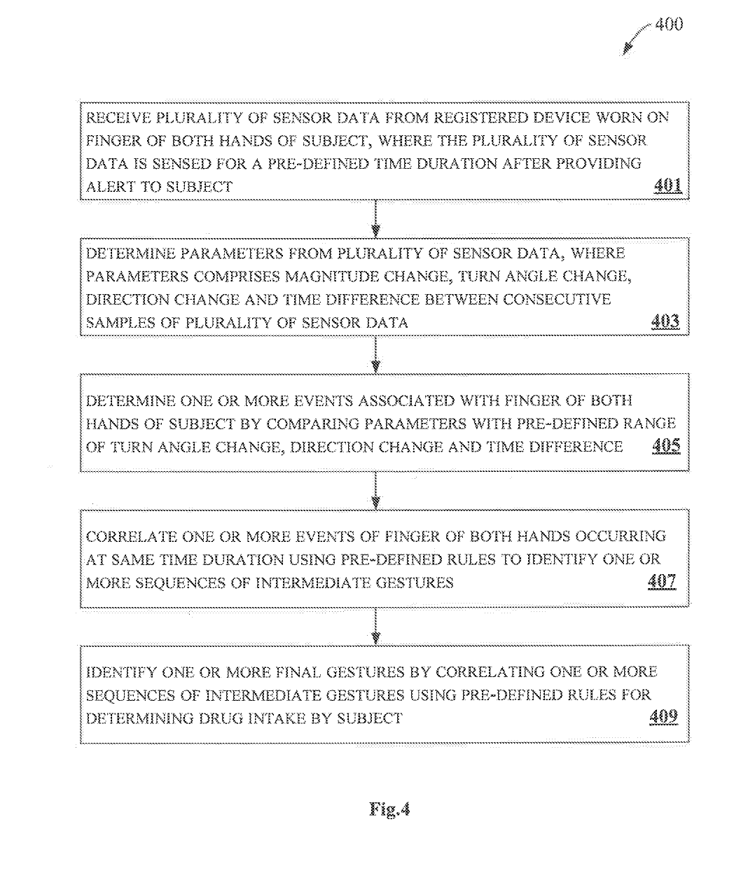

FIG. 4 illustrates a flowchart showing a method for determining drug intake by a subject 102 by monitoring gestures of the subject 102 in accordance with some embodiments of present disclosure.

As illustrated in FIG. 4, the method 400 comprises one or more blocks for determining drug intake by a subject. The method 400 may be described in the general context of computer executable instructions. Generally, computer executable instructions can include routines, programs, objects, components, data structures, procedures, modules, and functions, which perform particular functions or implement particular abstract data types.

The order in which the method 400 is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block 401, the gesture monitoring system 101 receives a plurality of sensor data from a registered device worn on a finger of both hands of a subject 102. The plurality of sensor data is sensed for a pre-defined time duration after providing an alert to the subject 102.

At block 403, the gesture monitoring system 101 determines parameters from the plurality of sensor data, wherein the parameters comprise magnitude change, turn angle change, direction change and time difference between consecutive samples of the plurality of sensor data.

At block 405, the gesture monitoring system 101 determines one or more events associated with the finger of both hands of the subject 102 by comparing the parameters with a pre-defined range of turn angle change, direction change and time difference.

At block 407, the gesture monitoring system 101 correlates the one or more events of the finger of both the hands occurring at same time duration using predefined rules to identify one or more sequences of intermediate gestures.

At block 409, the gesture monitoring system 101 identifies one or more final gestures by correlating the one or more sequences of intermediate gestures using the pre-defined rules for determining drug intake by the subject 102.

### Computing System

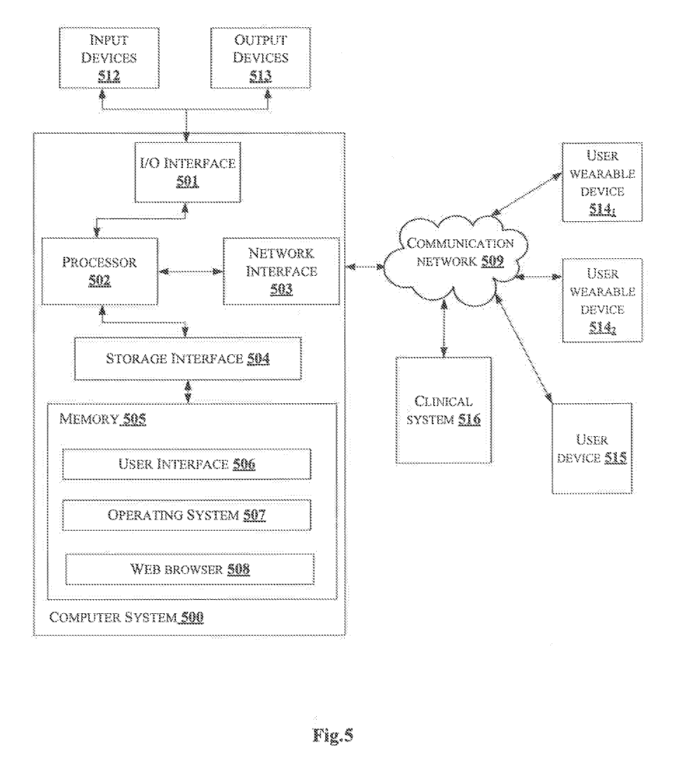

FIG. 5 illustrates a block diagram of an exemplary computer system 500 for implementing embodiments consistent with the present disclosure. In an embodiment, the computer system 500 is used to implement the gesture monitoring system 101. The computer system 500 may comprise a central processing unit (“CPU” or “processor”) 502. The processor 502 may comprise at least one data processor for determining drug intake by a subject by monitoring gestures of the subject. The processor 502 may include specialized processing units such as, integrated system (bus) controllers, memory management control units, floating point units, graphics processing units, digital signal processing units, etc.

The processor 502 may be disposed in communication with one or more input/output (I/O) devices (not shown) via I/O interface 501. The I/O interface 501 may employ communication protocols/methods such as, without limitation, audio, analog, digital, monoaural. RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using the I/O interface 501, the computer system 500 may communicate with one or more I/O devices. For example, the input device may be an antenna, keyboard, mouse, joystick. (infrared) remote control, camera, card reader, fax machine, dongle, biometric reader, microphone, touch screen, touchpad, trackball, stylus, scanner, storage device, transceiver, video device/source, etc. The output device may be a printer, fax machine, video display (e.g., cathode ray tube (CRT), liquid crystal display (LCD), light-emitting diode (LED), plasma, Plasma display panel (PDP), Organic light-emitting diode display (OLED) or the like), audio speaker, etc.

In some embodiments, the computer system 500 consists of a gesture monitoring system 101. The processor 502 may be disposed in communication with the communication network 509 via a network interface 503. The network interface 503 may communicate with the communication network 509. The network interface 503 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. The communication network 509 may include, without limitation, a direct interconnection, local area network (LAN), wide area network (WAN), wireless network (e.g., using Wireless Application Protocol), the Internet, etc. Using the network interface 503 and the communication network 509, the computer system 500 may communicate with a user wearable device 5141, a user wearable device 514₂, user device 515 and a clinical system 516. The network interface 503 may employ connection protocols include, but not limited to, direct connect, Ethernet (e.g., twisted pair 10/100/1000 Base T), transmission control protocol/internet protocol (TCP/IP), token ring, IEEE 802.11a/b/g/n/x, etc.

The communication network 509 includes, but is not limited to, a direct interconnection, an e-commerce network, a peer to peer (P2P) network, local area network (LAN), wide area network (WAN), wireless network (e.g., using Wireless Application Protocol), the Internet, Wi-Fi and such. The first network and the second network may either be a dedicated network or a shared network, which represents an association of the different types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the first network and the second network may include a variety of network devices, including routers, bridges, servers, computing devices, storage devices, etc.

In some embodiments, the processor 502 may be disposed in communication with a memory 505 (e.g., RAM, ROM, etc. not shown in FIG. 5) via a storage interface 504. The storage interface 504 may connect to memory 505 including, without limitation, memory drives, removable disc drives, etc., employing connection protocols such as, serial advanced technology attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 505 may store a collection of program or database components, including, without limitation, user interface 506, an operating system 507 etc. In some embodiments, computer system 500 may store user/application data 506, such as, the data, variables, records, etc., as described in this disclosure. Such databases may be implemented as fault-tolerant, relational, scalable, secure databases such as Oracle or Sybase.

The operating system 507 may facilitate resource management and operation of the computer system 500. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include Random Access Memory (RAM), Read-Only Memory (ROM), volatile memory, non-volatile memory, hard drives, CD ROMs, DVDs, flash drives, disks, and any other known physical storage media.

An embodiment of the present disclosure determines drug intake by the subject by identifying finger gestures of both hands of the subject.

An embodiment of the present disclosure analyses the natural gestures of the subject to confirm on the actual intake of the drug.

An embodiment of the present disclosure provides efficient determination of drug intake based on only scientific methods by having null dependence on any explicit user specific feedback.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor and a processor capable of processing and executing the queries. A non-transitory computer readable medium may comprise media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. Further, non-transitory computer-readable media comprise all computer-readable media except for a transitory. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as, an optical fiber, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory computer readable medium at the receiving and transmitting stations or devices. An “article of manufacture” comprises non-transitory computer readable medium, hardware logic, and/or transmission signals in which code may be implemented. A device in which the code implementing the described embodiments of operations is encoded may comprise a computer readable medium or hardware logic. Of course, those skilled in the art will recognize that many modifications may be made to this configuration without departing from the scope of the invention, and that the article of manufacture may comprise suitable information bearing medium known in the art.

The terms “an embodiment”, “embodiment”, “embodiments”, “the embodiment”, “the embodiments”, “one or more embodiments”. “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIG. 4 show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processing unit or by distributed processing units.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference | |
|---|---|
| Number | Description |
| 100 | Environment |
| 101 | Gesture monitoring system |
| 102 | User |
| 103 | User wearable devices |
| 105 | User device |
| 107 | Clinical system |
| 109 | Communication network |
| 111 | I/O interface |
| 113 | Memory |
| 115 | Processor |
| 117 | Accelerometer |
| 119 | Magnetometer |
| 121 | Camera |
| 123 | Vibrator |
| 200 | Data |
| 201 | Sensor data |
| 203 | Prescription data |
| 205 | User device registration data |
| 207 | Events data |
| 209 | Gesture data |
| 211 | Other data |
| 213 | Modules |
| 215 | Receiving module |
| 217 | Parameters Determining module |
| 219 | Events determining module |
| 221 | Correlation module |
| 223 | Drug intake determination module |
| 225 | Other modules |

What is claimed is:

1. A method for determining drug intake by a subject by monitoring gestures of the subject, the method comprising:
receiving, by a gesture monitoring system, a plurality of sensor data from a registered device worn on a finger of both hands of a subject, wherein the plurality of sensor data is sensed for a pre-defined time duration after providing an alert to the subject;
determining, by the gesture monitoring system, parameters from the plurality of sensor data, wherein the parameters comprises magnitude change, turn angle change, direction change and time difference between consecutive samples of the plurality of sensor data;
determining, by the gesture monitoring system, one or more events associated with the finger of both hands of the subject by comparing the parameters with a pre-defined range of turn angle change, direction change and time difference;
correlating, by the gesture monitoring system, the one or more events of the finger of both the hands occurring at same time duration using predefined rules to identify one or more sequences of intermediate gestures; and
identifying, by the gesture monitoring system, one or more final gestures by correlating the one or more sequences of intermediate gestures using the pre-defined rules for determining drug intake by the subject.

2. The method as claimed in claim 1, wherein the alert is provided to the subject by at least one of, through a registered communication device and through the registered device worn by the subject.

3. The method as claimed in claim 1, wherein the plurality of sensor data received is filtered by the registered device.

4. The method as claimed in claim 1, wherein the plurality of sensor data comprises coordinates details and orientation information associated with the finger of both hands of the subject.

5. The method as claimed in claim 1, wherein the one or more gestures for drug intake are pre-defined and stored in the gesture monitoring system.

6. The method as claimed in claim 1, wherein the one or more events comprises clockwise motion, anti-clockwise motion, angular motion, rotary motion, tilt movement and linear motion of both hands of the subject.

7. The method as claimed in claim 1, wherein the one or more final gestures comprises at least one of tearing a drug strip, sliding of drug from strip, taking the drug to mouth, placing the drug in the mouth, holding a container, tilting the container, opening and closing of a tap, holding a container of water and taking the container of water to mouth.

8. The method as claimed in claim 1, wherein the one or more sequences of intermediate gestures are identified based on an order of time duration.

9. A gesture monitoring system for determining drug intake by a subject by monitoring gestures of the subject, comprising:
a processor; and
a memory communicatively coupled to the processor, wherein the memory stores processor instructions, which, on execution, causes the processor to:
receive a plurality of sensor data from a registered device worn on a finger of both hands of a subject, wherein the plurality of sensor data is sensed for a pre-defined time duration after providing an alert to the subject;
determine parameters from the plurality of sensor data, wherein the parameters comprise magnitude change, turn angle change, direction change and time difference between consecutive samples of the plurality of sensor data;
determine one or more events associated with the finger of both hands of the subject by comparing the parameters with a pre-defined range of turn angle change, direction change and time difference;
correlate the one or more events of the finger of both the hands occurring at same time duration using predefined rules to identify one or more sequences of intermediate gestures; and
identify one or more final gestures by correlating the one or more sequences of intermediate gestures using the pre-defined rules for determining drug intake by the subject.

10. The gesture monitoring system as claimed in claim 9, wherein the alert is provided to the subject by at least one of, through a registered communication device and through the registered device worn by the subject.

11. The gesture monitoring system as claimed in claim 9, wherein the plurality of sensor data received is filtered by the registered device.

12. The gesture monitoring system as claimed in claim 9, wherein the plurality of sensor data comprises coordinates details and orientation information associated with the finger of both hands of the subject.

13. The gesture monitoring system as claimed in claim 9, wherein the one or more gestures for drug intake are pre-defined and stored in the gesture monitoring system.

14. The gesture monitoring system as claimed in claim 9, wherein the one or more events comprises clockwise motion, anti-clockwise motion, angular motion, rotary motion, tilt movement and linear motion of both hands of the subject.

15. The gesture monitoring system as claimed in claim 9, wherein the one or more final gestures comprises at least one of tearing a drug strip, sliding of drug from strip, taking the drug to mouth, placing the drug in the mouth, holding a container, tilting the container, opening and closing of a tap, holding a container of water and taking the container of water to mouth.

16. The gesture monitoring system as claimed in claim 9, wherein the one or more sequences of intermediate gestures are identified based on an order of time duration.

17. A non-transitory computer readable medium including instruction stored thereon that when processed by at least one processor cause a gesture monitoring system to perform operation comprising:
receiving a plurality of sensor data from a registered device worn on a finger of both hands of a subject, wherein the plurality of sensor data is sensed for a pre-defined time duration after providing an alert to the subject;
determining parameters from the plurality of sensor data, wherein the parameters comprise magnitude change, turn angle change, direction change and time difference between consecutive samples of the plurality of sensor data;
determining one or more events associated with the finger of both hands of the subject by comparing the parameters with a pre-defined range of turn angle change, direction change and time difference;
correlating the one or more events of the finger of both the hands occurring at same time duration using predefined rules to identify one or more sequences of intermediate gestures; and
identifying one or more final gestures by correlating the one or more sequences of intermediate gestures using the pre-defined rules for determining drug intake by the subject.

18. The medium as claimed in claim 17, wherein the instruction causes the processor to provide alert to the subject by at least one of, through a registered communication device and through the registered device worn by the subject.

19. The medium as claimed in claim 17, wherein the plurality of sensor data received is filtered by the registered device.

20. The medium as claimed in claim 17, wherein the plurality of sensor data comprises coordinates details and orientation information associated with the finger of both hands of the subject.

21. The medium as claimed in claim 17, wherein the one or more gestures for drug intake are pre-defined and stored in the gesture monitoring system.

22. The medium as claimed in claim 17, wherein the one or more events comprises clockwise motion, anti-clockwise motion, angular motion, rotary motion, tilt movement and linear motion of both hands of the subject.

23. The medium as claimed in claim 17, wherein the one or more final gestures comprises at least one of tearing a drug strip, sliding of drug from strip, taking the drug to mouth, placing the drug in the mouth, holding a container, tilting the container, opening and closing of a tap, holding a container of water and taking the container of water to mouth.

24. The medium as claimed in claim 17, wherein the instruction causes the processor to identify one or more sequences of intermediate gestures based on an order of time duration.
